(19) United States
(12) Patent Application Publication
(10) Pub. No.: US 2025/0388845 A1
(43) Pub. Date: Dec. 25, 2025

(54) OPTICAL EXAMINING SYSTEM FOR CAPTURING MULTILAYER MICROSCOPIC IMAGES INSIDE CELL CULTURE BOTTLE

(71) Applicant: CellFabs Inc., Tainan City (TW)

(72) Inventors: Yun-Shu Chiou, Tainan City (TW); Tzu-Yang Huang, Tainan City (TW); Shang-Chih Lin, Tainan City (TW)

(73) Assignee: CellFabs Inc., Tainan City (TW)

(21) Appl. No.: 18/749,387

(22) Filed: Jun. 20, 2024

(51) Int. Cl. C12M 1/34; C12M 1/00; C12M 1/12; C12M 1/24; C12M 1/36

(52) U.S. Cl. CPC C12M 41/36; C12M 23/08; C12M 23/22; C12M 37/00; C12M 41/48

(57) ABSTRACT

An optical examining system for capturing multilayer microscopic images includes a cell culture bottle with parallel layers of cell culture beds arranged internally. An external surface of the cell culture bottle forms multiple concave-shaped imaging wells, each well having a bottom plate at varying depths. Each of the bottom plates is close to one of the cell culture beds of different heights. An optical examining device is equipped with an examining platform for placing the cell culture bottle. Above the examining platform, a movable microscope is provided, and extending downward from the microscope is an objective lens that enters the imaging wells. This allows the objective lens to capture microscopic images of multiple layers of the cell culture beds at positions near the bottom plates. The system aims to obtain highly accurate assessments of cell quantity and quality.

### BACKGROUND OF THE DISCLOSURE

### 1. Field of the Disclosure

The present invention relates generally to an automated cellular microscopy imaging apparatus, and more particularly to an optical examining system for capturing multilayer microscopic images inside a cell culture bottle, characterized by high cellular recognition rates and the capability to prevent contamination of the cells.

### 2. Description of the Prior Art

In conventional cell culture methods, cells are typically placed inside cell culture bottles and immersed in culture medium for preservation in an environment conducive to cell growth. After successful cultivation, all cells are removed from the culture bottle to confirm their growth status. Typically, a small number of cells are first extracted from the culture bottle and placed on slides for manual observation or imaging under a microscope to assess cell viability and quality. However, it is evident upon closer examination of this conventional method that it has some shortcomings. The main issue lies in the fact that the manual extraction of cells may lead to contamination of the culture bottle, resulting in failed cell cultivation. Therefore, many industry practitioners desire the ability to assess cell growth directly outside a culture bottle. However, direct imaging outside the bottle yields results inferior to microscopy, leading to decreased accuracy in cell recognition. Thus, the challenge addressed by this invention is how to improve the detection of cell growth status from outside of the bottle.

In light of these challenges, the inventor, drawing upon years of experience in the manufacturing, development, and design of related products, meticulously designed and assessed solutions to the aforementioned objectives, finally achieving a truly viable invention.

### SUMMARY OF THE DISCLOSURE

The technical problem that the present invention aims to solve is to address the aforementioned shortcomings of existing technology by providing an optical examining system for capturing multilayer microscopic images inside a cell culture bottle.

A cell culture bottle has a plurality of cell culture beds of different heights arranged in parallel therein, and the cell culture bottle has a plurality of concave-shaped imaging wells formed on an external surface thereof, wherein each of the concave-shaped imaging wells has a bottom plate of a different depth. Each of the bottom plates is transparent and is close to one of the cell culture beds of different heights. An optical examining device is provided with an examining platform for placing the cell culture bottle thereon, and a microscope provided above the examining platform in a movable manner. The microscope has an objective lens extending downward, and the objective lens is adapted to extend into the concave-shaped imaging wells, whereby the objective lens captures microscopic images of the cell culture beds through the bottom plates.

The technical problem that the present invention aims to solve is to address the aforementioned shortcomings of existing technology and further provide an optical examining system for capturing multilayer microscopic images inside a cell culture bottle.

A cell culture bottle has ten cell culture beds of different heights arranged in parallel therein, wherein the cell culture beds comprises a first cell culture bed to a tenth cell culture bed, numbered according to their heights; an external surface the cell culture bottle is recessed to form at least one first imaging shaft, at least one second imaging shaft, and at least one third imaging shaft; a bottom of the first imaging shaft is at the first cell culture bed, a bottom of the second imaging shaft is at the fifth cell culture bed, and a bottom of the third imaging shaft is at the tenth cell culture bed. An optical examining device, which is provided with an examining platform for placing the cell culture bottle thereon, and a microscope provided above the examining platform in a movable manner, wherein the microscope has an objective lens extending downward, and the objective lens is adapted to extend into the first imaging shaft, the second imaging shaft, and the third imaging shaft, whereby the objective lens captures microscopic images of the cell culture beds.

In an embodiment, the optical examining device is equipped with a light source positioned beneath the examination platform, and the light source is planar to accommodate placement of the cell culture bottle thereon.

In an embodiment, the light source has an automatic dimming function; the light source becomes brighter as the objective lens moves farther from the examining platform.

In an embodiment, the optical examining device is provided inside a box, and the box is provided with a plurality of UV sterilization lamps therein; when the cell culture bottle is not placed on the examining platform, the UV sterilization lamps are activated.

In an embodiment, the optical examining device fixes the examining platform with a bottom of a stand; a motor is fixed at a top of the stand, and the motor is connected to a screw rod which stands perpendicular to the examining platform; the screw rod engages to drive a lifting support in a manner that the lifting support moves the microscope up and down.

In an embodiment, the optical examining device is provided with a horizontal displacement mechanism at the lifting support, and the microscope is fixed by the horizontal displacement mechanism; the horizontal displacement mechanism comprises a left-right displacement slide rail and a front-back displacement slide rail.

In an embodiment, when the microscope captures the microscopic images of one of the cell culture beds, a distance between the objective lens and the cell culture bed being shot ranges from 2 to 10 mm.

In an embodiment, the optical examining device has an AI analysis unit, which analyzes the microscopic images of the cell culture bed being shot by using an image recognition method; when the AI analysis unit automatically determines that a cell amount is sufficient, the cell culture bottle is sent to a centrifugal harvesting station to extract cell products.

In an embodiment, the cell culture bottle is retrieved from a cell culture chamber and placed on a conveyor belt, and the conveyor belt transports the cell culture bottle to the optical examining device, where the cell culture bottle is gripped and placed on the examining platform by a robotic arm; if the AI analysis unit automatically determines that cell amount is insufficient, the cell culture bottle is sent to a medium replacement station, and then transported by the conveyor belt back to the cell culture chamber for storage.

The first main purpose of the present invention is to allow the microscope to perform forward, backward, left, right, and up and down displacement according to the set automatic program, so that the objective lens can sequentially align with each concave-shaped imaging well. Then, the objective lens extends into the aforementioned concave-shaped imaging well, allowing the objective lens to complete the capture of microscopic images of one of the multi-layered cell culture beds near the bottom plate. Subsequently, multiple microscopic images are analyzed to obtain highly precise assessments of cell quantity and cell quality.

The second main purpose of the present invention is to optimize the design of cell culture bottle by setting up ten layers of cell culture beds, which is achieved through a comprehensive evaluation of space utilization within the bottle and the amount of culture medium added. This optimization aims to maximize the cell culture yield, cell quality, and cell culture efficiency. Furthermore, by displacing the objective lens between the first imaging shaft, the second imaging shaft, and the third imaging shaft, and respectively capturing images of the cell culture beds at the first layer, the fifth layer, and the tenth layer, precise analysis of cell culture quantity and quality can be achieved with the minimum number of captures.

Other objectives, advantages, and novel features of the present invention will become more apparent from the following detailed description and accompanying drawings.

### BRIEF DESCRIPTION OF THE DRAWINGS

The present invention will be best understood by referring to the following detailed description of one illustrative embodiment in conjunction with the accompanying drawings, in which

FIG. 1 is a perspective view of a cell culture bottle and an optical examining device of the present invention;

FIG. 2 is a sectional view showing the objective lens of the present invention approaching the cell culture bottle;

FIG. 3 is a perspective view showing the cell culture bottle being placed on the examining platform;

FIG. 4 is a perspective view showing the operation of approaching the objective lens to the cell culture bottle;

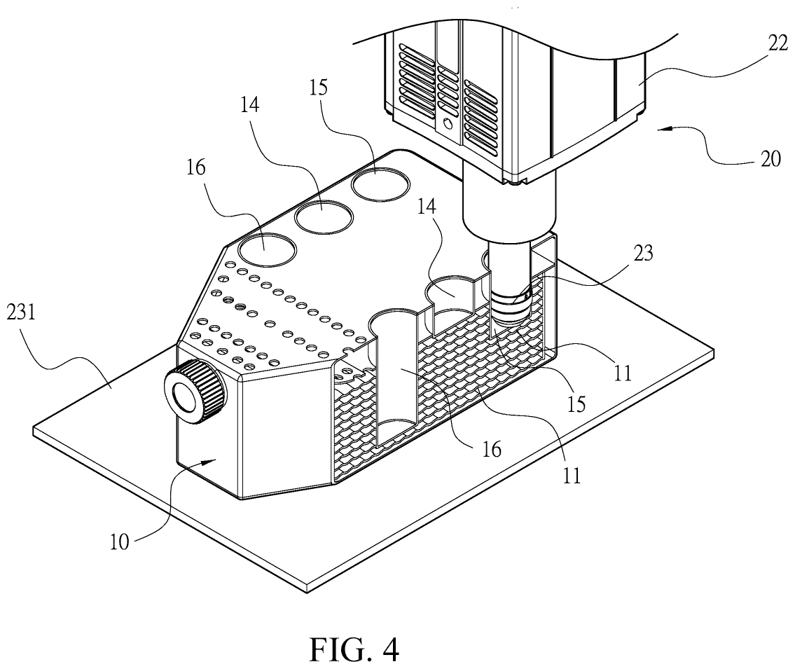

FIG. 5 is another perspective view showing the operation of approaching the objective lens to the cell culture bottle;

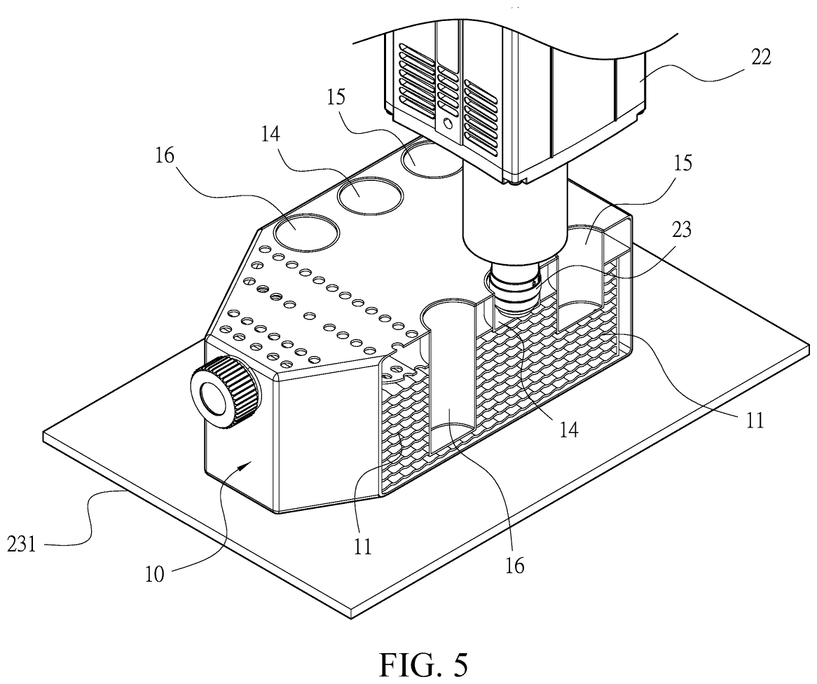

FIG. 6 is another sectional view showing the objective lens of the present invention approaching the cell culture bottle;

FIG. 7 is a schematic view showing the operation of the optical examining device as seen from one side;

FIG. 8 is a schematic view showing the operation of the optical examining device as seen from the front; and

FIG. 9 is a flowchart showing the operation of examining the cell culture bottle in an optical manner.

### DETAILED DESCRIPTION

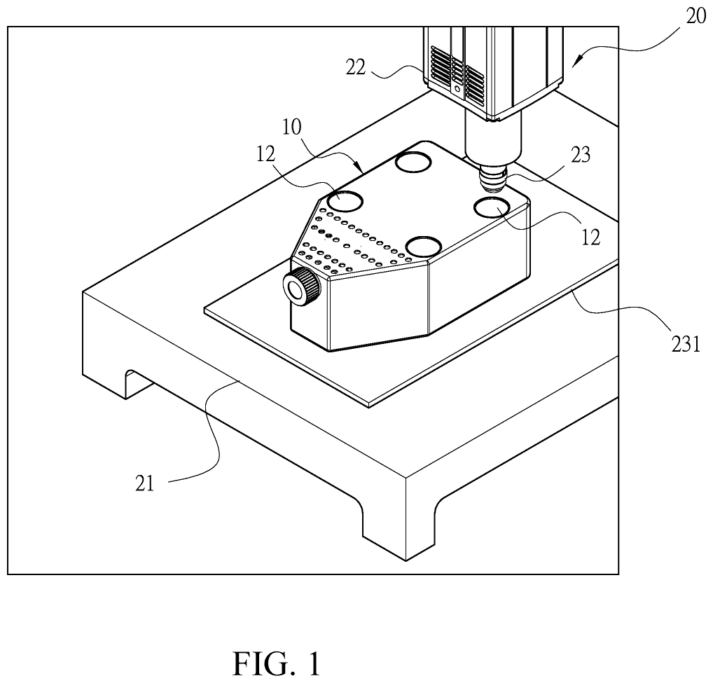

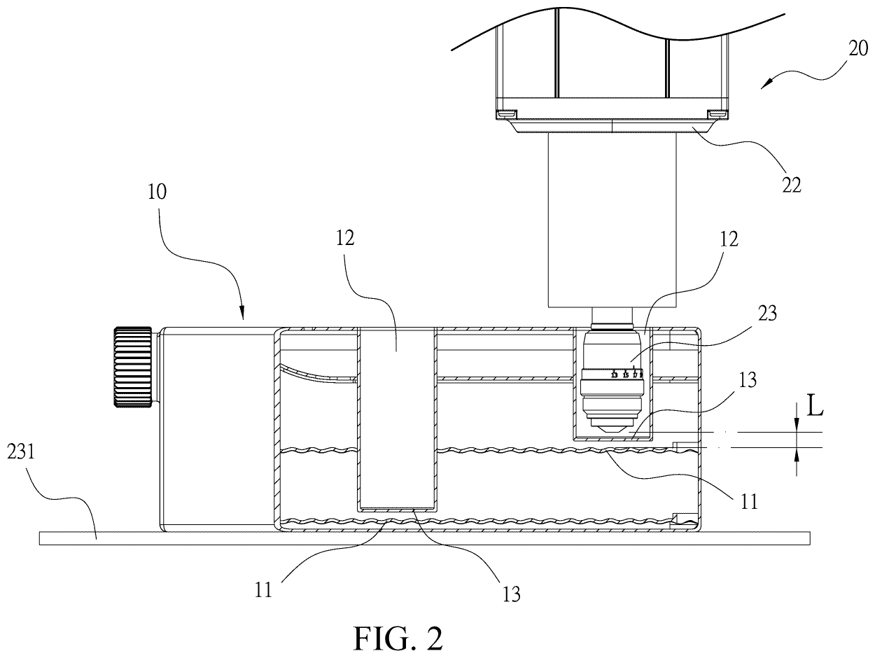

Please refer to FIG. 1 and FIG. 2 first, which illustrate an optical examining system for capturing multilayer microscopic images inside a cell culture bottle. The optical examining system includes a cell culture bottle 10 and an optical examining device 20. The cell culture bottle 10 is internally equipped with multiple layers of cell culture beds 11 of different heights, and an external surface of the cell culture bottle 10 forms a plurality of concave-shaped imaging wells 12. Each of the concave-shaped imaging wells 12 has a bottom plate 13 of different depths, with multiple transparent bottom plates 13 positioned near the cell culture beds 11 of different heights. The optical examining device 20 is equipped with an examining platform 21 for placing the cell culture bottle 10. A microscope 22, which can be vertically displaced above the examining platform 21, extends downward with an objective lens 23. The optical examining device 20 has a planar light source 231 positioned below the examining platform 21, wherein the planar light source 231 provides direct placement for the cell culture bottle 10. In summary, after the cell culture bottle 10 is placed on the examining platform 21, the microscope 22 moves according to a preset automatic program. This movement includes forward, backward, left, right, and up and down displacement relative to the optical examining device 20, where the back of the optical examining device 20 is considered the rear, and the examining platform 21 is positioned below the optical examining device 20. This allows the objective lens 23 to sequentially align with each of the concave-shaped imaging wells 12. Then, the objective lens 23 extends into each of the concave-shaped imaging wells 12, positioning itself near the desired area of one of the cell culture beds 11 to achieve the optimal microscopic imaging effect. The distance (L) between the objective lens 23 and the cell culture bed 11 being shot ranges from 2 to 10 millimeters (mm). Through multiple movements and extensions of the objective lens 23, microscopic images of multiple layers of the cell culture bed 11 are captured near the bottom plate 13. These microscopic images are then analyzed to obtain highly accurate assessments of cell quantity and cell quality.

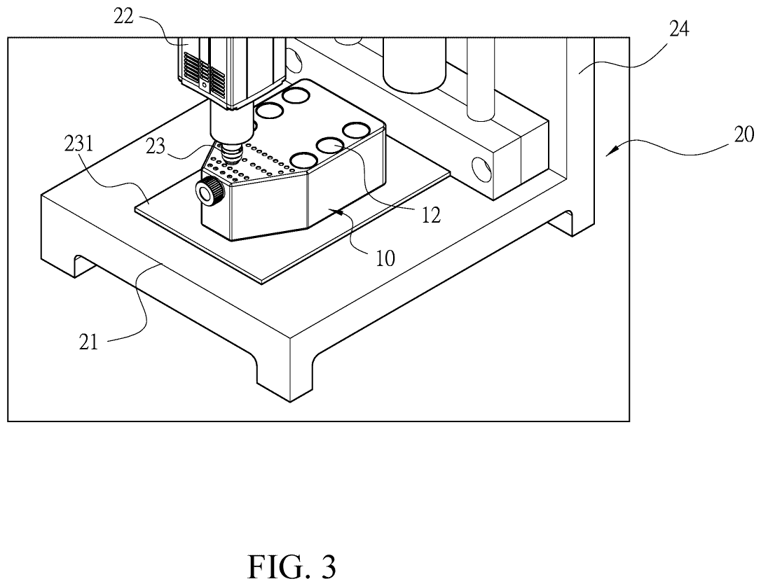

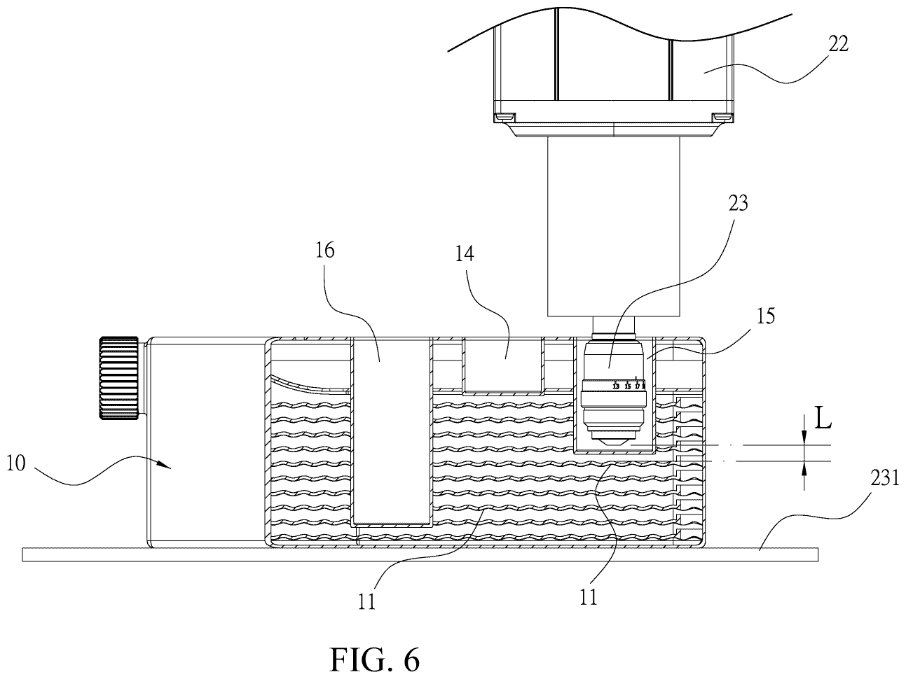

The embodiments are further explained below. FIG. 3 and FIG. 6 illustrate an optical examining system for capturing multilayer microscopic images inside a cell culture bottle. As shown in the drawings, a cell culture bottle 10 is internally equipped with ten cell culture beds 11 of different heights, and an external surface of the cell culture bottle 10 forms at least one first imaging shaft 14, at least one second imaging shaft 15, and at least one third imaging shaft 16. For ease of explanation, the cell culture beds 11 includes a first cell culture bed to a tenth cell culture bed, numbered according to their heights. A bottom of the first imaging shaft 14 is located at the first cell culture bed 11, a bottom of the second imaging shaft 15 is located at the fifth cell culture bed 11, and a bottom of the third imaging shaft 16 is located at the tenth cell culture bed 11. An optical examining device 20 is equipped with an examining platform 21 for placing the cell culture bottle 10. Positioned above the examining platform 21 is a microscope 22, which extends downward with an objective lens 23. The optical examining device 20 has a planar light source 231 positioned below the examining platform 21, wherein the light source 231 provides direct placement for the cell culture bottle 10. The light source 231 has an automatic dimming function, where the brightness increases as the distance between the objective lens 23 and the examining platform 21 increases, thus providing sufficient light for the microscope 22. In summary, after the cell culture bottle 10 is placed on the examining platform 21, the microscope 22 moves according to a preset automatic program. This movement includes forward, backward, left, right, and up and down displacement relative to the optical examining device 20, which corresponds to displacement in an XYZ coordinate system. This allows the objective lens 23 to sequentially align with the first imaging shaft 14. Then, the objective lens 23 extends into the first imaging shaft 14 to capture microscopic images of the first cell culture bed 11. After retracting the objective lens 23, the same process is repeated for the second imaging shaft 15 and the third imaging shaft 16, capturing microscopic images of the fifth and tenth cell culture beds 11, respectively. This achieves the optimal microscopic imaging effect, where the objective lens 23 sequentially extends into the first imaging shaft 14, the second imaging shaft 15, and the third imaging shaft 16 to capture microscopic images of multiple cell culture beds 11. By capturing images of the first, fifth, and tenth cell culture beds 11 sequentially, not only can the cultivation status of multiple cell culture beds 11 be determined, avoiding misjudgment due to better growth in single one of the cell culture beds 11, but comprehensive analysis of multiple microscopic images can also be conducted to obtain highly accurate assessments of cell quantity and cell quality.

Furthermore, the design of ten cell culture beds 11 in the cell culture bottle 10 is the result of a comprehensive evaluation of space utilization within the bottle 10 and the amount of culture medium added, aiming to achieve optimal design for cell culture quantity, cell quality, and cell culture efficiency. Subsequently, by moving the objective lens 23 between the first imaging shaft 14, the second imaging shaft 15, and the third imaging shaft 16, and capturing images of the first, third, and fifth cell culture beds 11 respectively, precise analysis of cell culture quantity and quality can be achieved with the minimum number of captures.

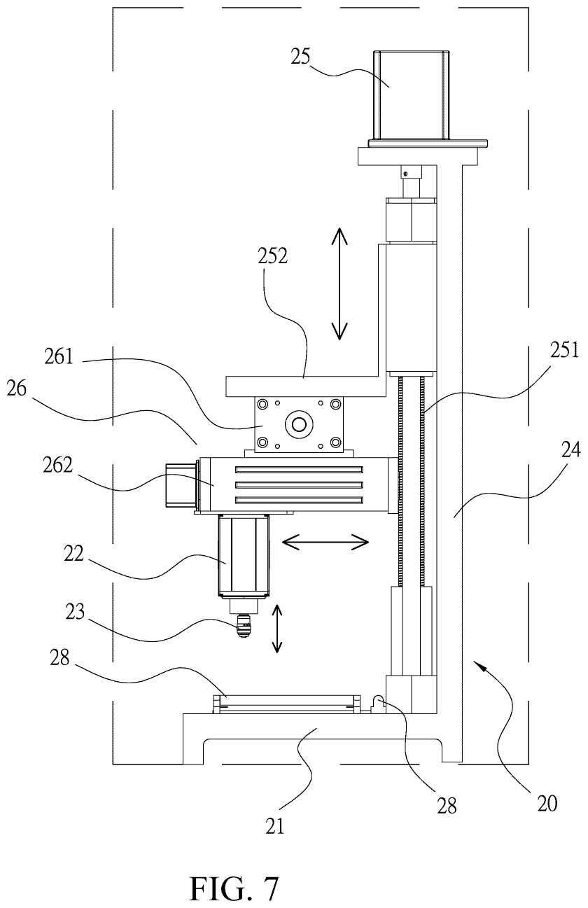

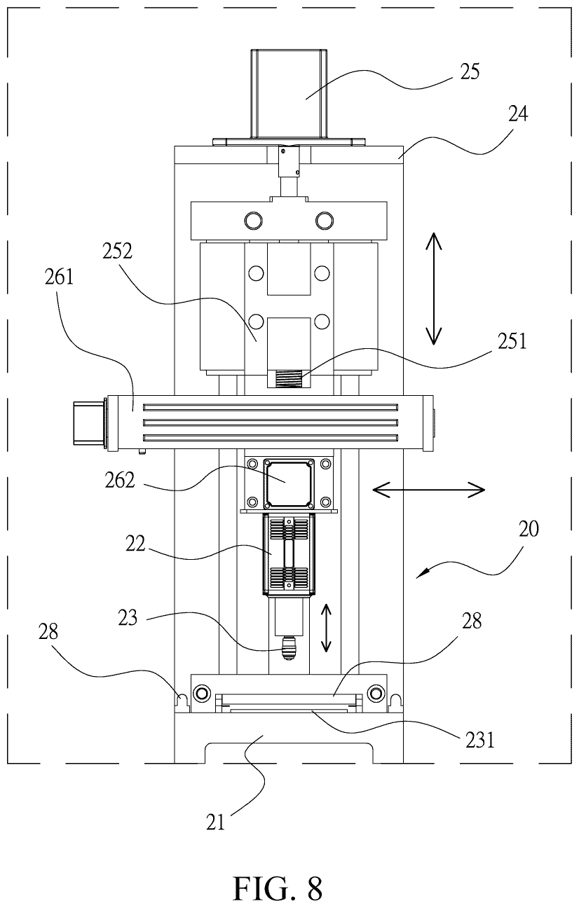

As shown in FIG. 6, FIG. 7, and FIG. 8, The optical examining device 20 is fixed at a bottom of a stand 24, with a top of the stand 24 has a motor 25 secured thereto. The motor 25 is connected to a screw rod 251, which is vertically aligned with the examining platform 21. The screw rod 251 engages with a lifting support 252, which in turn is connected to the microscope 22 to enable vertical displacement. Additionally, the optical examining device 20 is equipped with a horizontal displacement mechanism 26 at the lifting support 252, which securely holds the microscope 22. The horizontal displacement mechanism 26 consists of a left-right displacement slide rail 261 and a front-back displacement slide rail 262. This mechanism enables the microscope 22 to move horizontally, with the front-back displacement slide rail 262 facilitating forward and backward movement. This allows the objective lens 23 to align and extend into the first imaging shaft 14, the second imaging shaft 15, and the third imaging shaft 16 for the purpose of capturing microscopic images of cells. When the microscope 22 captures microscopic images of one of the cell culture beds 11, the distance (L) between the objective lens 23 and the cell culture bed 11 ranges from 2 to 10 millimeters (mm), ensuring optimal imaging results.

As shown in FIG. 7 and FIG. 8, the optical examining device 20 is housed within a box 27, which provides access for placing and retrieving the cell culture bottle 10. Inside the box 27, there are a plurality of UV sterilization lamps 28. These UV sterilization lamps 28 are activated to sterilize the cell culture bottle 10 when it is not placed on the examining platform 21. Through the barrier and reflection of ultraviolet light provided by the box 27, the entire optical examining device 20 can maintain a sterile environment, effectively reducing the risk of contamination to the cell culture bottle 10.

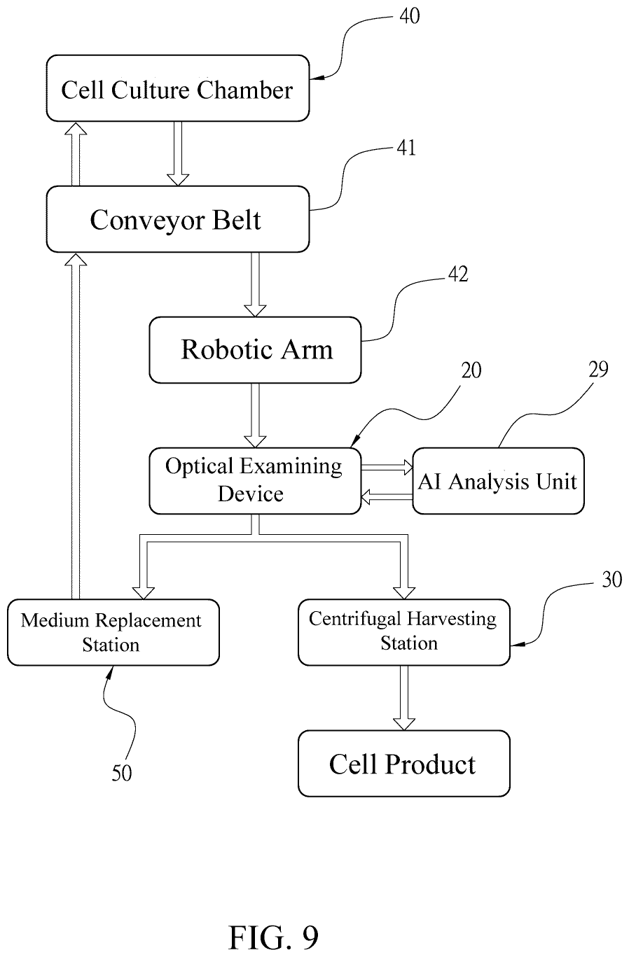

Furthermore, as shown in FIG. 9, the optical examining device 20 is equipped with an AI analysis unit 29, which analyzes the microscopic images of the cell culture beds 11 through an image recognition method. When the AI analysis unit 29 automatically determines that the cell amount is deemed sufficient, the cell culture bottle 10 is sent to a centrifugal harvesting station 30 to retrieve cell products. Additionally, the cell culture bottle 10 is initially taken from a cell culture chamber 40 and placed on a conveyor belt 41. The conveyor belt 41 transports the cell culture bottle 10 to the optical examining device 20, where it is held in place on the examining platform 21 by a robotic arm 42. When the optical examining device 20 detects the presence of the cell culture bottle 10, it automatically initiates the imaging process. Furthermore, if the AI analysis unit 29 determines that the cell amount is insufficient, the cell culture bottle 10 is sent to a medium replacement station 50 to replace the culture medium. Subsequently, the conveyor belt 41 transports it back to the cell culture chamber 40 for storage. This automated process aims to save labor and reduce the risk of cell contamination. Moreover, by utilizing the AI analysis unit 29 to enhance the precision of image recognition, the automated system can make more stable decisions for the next steps, whether it be retrieving cell products or replacing the culture medium, thus achieving optimal efficiency.

It should be realized that the above description is only some preferred embodiments of the present invention and should not be deemed as limitations of implementing the present invention. All substantially equivalent variations and modifications which employ the concepts disclosed in this specification and the appended claims should fall within the scope of the present invention.

What is claimed is:

1. An optical examining system, comprising:
a cell culture bottle, which has a plurality of cell culture beds of different heights arranged in parallel therein, and the cell culture bottle has a plurality of concave-shaped imaging wells formed on an external surface thereof, wherein each of the concave-shaped imaging wells has a bottom plate of a different depth; each of the bottom plates is transparent and is close to one of the cell culture beds of different heights; and
an optical examining device, which is provided with an examining platform for placing the cell culture bottle thereon, and a microscope provided above the examining platform in a movable manner, wherein the microscope has an objective lens extending downward, and the objective lens is adapted to extend into the concave-shaped imaging wells, whereby the objective lens captures microscopic images of the cell culture beds through the bottom plates.

2. The optical examining system of claim 1, wherein the optical examining device is equipped with a light source positioned beneath the examination platform, and the light source is planar to accommodate placement of the cell culture bottle thereon.

3. The optical examining system of claim 2, wherein the light source has an automatic dimming function; the light source becomes brighter as the objective lens moves farther from the examining platform.

4. The optical examining system of claim 1, wherein the optical examining device is provided inside a box, and the box is provided with a plurality of UV sterilization lamps therein; when the cell culture bottle is not placed on the examining platform, the UV sterilization lamps are activated.

5. The optical examining system of claim 1, wherein the optical examining device fixes the examining platform with a bottom of a stand; a motor is fixed at a top of the stand, and the motor is connected to a screw rod which stands perpendicular to the examining platform; the screw rod engages to drive a lifting support in a manner that the lifting support moves the microscope up and down.

6. The optical examining system of claim 5, wherein the optical examining device is provided with a horizontal displacement mechanism at the lifting support, and the microscope is fixed by the horizontal displacement mechanism; the horizontal displacement mechanism comprises a left-right displacement slide rail and a front-back displacement slide rail.

7. The optical examining system of claim 1, wherein, when the microscope captures the microscopic images of one of the cell culture beds, a distance between the objective lens and the cell culture bed being shot ranges from 2 to 10 millimeters.

8. The optical examining system of claim 1, wherein the optical examining device has an AI analysis unit, which analyzes the microscopic images of the cell culture bed being shot by using an image recognition method; when the AI analysis unit automatically determines that a cell amount is sufficient, the cell culture bottle is sent to a centrifugal harvesting station to extract cell products.

9. The optical examining system of claim 8, wherein the cell culture bottle is retrieved from a cell culture chamber and placed on a conveyor belt, and the conveyor belt transports the cell culture bottle to the optical examining device, where the cell culture bottle is gripped and placed on the examining platform by a robotic arm; if the AI analysis unit automatically determines that cell amount is insufficient, the cell culture bottle is sent to a medium replacement station, and then transported by the conveyor belt back to the cell culture chamber for storage.

10. An optical examining system, comprising:
a cell culture bottle, which has ten cell culture beds of different heights arranged in parallel therein, wherein the cell culture beds comprises a first cell culture bed to a tenth cell culture bed, numbered according to their heights; an external surface the cell culture bottle is recessed to form at least one first imaging shaft, at least one second imaging shaft, and at least one third imaging shaft; a bottom of the first imaging shaft is at the first cell culture bed, a bottom of the second imaging shaft is at the fifth cell culture bed, and a bottom of the third imaging shaft is at the tenth cell culture bed; and
an optical examining device, which is provided with an examining platform for placing the cell culture bottle thereon, and a microscope provided above the examining platform in a movable manner, wherein the microscope has an objective lens extending downward, and the objective lens is adapted to extend into the first imaging shaft, the second imaging shaft, and the third imaging shaft, whereby the objective lens captures microscopic images of the cell culture beds.

11. The optical examining system of claim 10, wherein the optical examining device is equipped with a light source positioned beneath the examination platform, and the light source is planar to accommodate placement of the cell culture bottle thereon.

12. The optical examining system of claim 11, wherein the light source has an automatic dimming function; the light source becomes brighter as the objective lens moves farther from the examining platform.

13. The optical examining system of claim 10, wherein the optical examining device is provided inside a box, and the box is provided with a plurality of UV sterilization lamps therein; when the cell culture bottle is not placed on the examining platform, the UV sterilization lamps are activated.

14. The optical examining system of claim 10, wherein the optical examining device fixes the examining platform with a bottom of a stand; a motor is fixed at a top of the stand, and the motor is connected to a screw rod which stands perpendicular to the examining platform; the screw rod engages to drive a lifting support in a manner that the lifting support moves the microscope up and down.

15. The optical examining system of claim 14, wherein the optical examining device is provided with a horizontal displacement mechanism at the lifting support, and the microscope is fixed by the horizontal displacement mechanism; the horizontal displacement mechanism comprises a left-right displacement slide rail and a front-back displacement slide rail.

16. The optical examining system of claim 10, wherein, when the microscope captures the microscopic images of one of the cell culture beds, a distance between the objective lens and the cell culture bed being shot ranges from 2 to 10 millimeters.

17. The optical examining system of claim 10, wherein the optical examining device has an AI analysis unit, which analyzes the microscopic images of the cell culture bed being shot by using an image recognition method; when the AI analysis unit automatically determines that a cell amount is sufficient, the cell culture bottle is sent to a centrifugal harvesting station to extract cell products.

18. The optical examining system of claim 17, wherein the cell culture bottle is retrieved from a cell culture chamber and placed on a conveyor belt, and the conveyor belt transports the cell culture bottle to the optical examining device, where the cell culture bottle is gripped and placed on the examining platform by a robotic arm; if the AI analysis unit automatically determines that cell amount is insufficient, the cell culture bottle is sent to a medium replacement station, and then transported by the conveyor belt back to the cell culture chamber for storage.
